(19) United States
(12) Patent Application Publication
(10) Pub. No.: US 2023/0159440 A1
(43) Pub. Date: May 25, 2023

(54) COMPOSITIONS AND METHODS TO INHIBIT FOULING OF AMMONIUM SALTS

(71) Applicant: ECOLAB USA INC., St. Paul, MN (US)

(72) Inventors: Shiguang LI, Shanghai (CN); Peter Jian Jun SUN, Beijing (CN); Qing Qing YUAN, Shanghai (CN)

(73) Assignee: ECOLAB USA INC., St. Paul, MN (US)

(21) Appl. No.: 17/917,538

(22) PCT Filed: Apr. 8, 2020
(86) PCT No.: PCT/CN2020/083628

§ 371 Date: Oct. 6, 2022

(51) Int. Cl. C07C 253/32

(52) U.S. Cl. CPC C07C 253/32

(57) ABSTRACT

Disclosed are antifoulant compounds or compositions used for inhibiting or reducing the deposition of ammonium salts.

### FIELD OF THE INVENTION

The application is directed at inhibiting or reducing fouling of processes by ammonium salts.

### BACKGROUND

A number of industrial processes such as the production of (meth)acrylonitrile, hydrogen cyanide or the treatment of coke oven gas create industrial process streams containing residual ammonia. Recovery and reuse of the residual ammonia increases the economic viability of these and other ammonia-producing processes.

Residual ammonia can be recovered from industrial processes with acid such as sulfuric acid in the form of ammonium sulfate salts. However, ammonium sulfate and other ammonium salts can precipitate and deposit onto equipment surfaces causing fouling. Fouling of the equipment such as heat exchangers, reboilers, pipes, condensers, columns and the like are a burden on production and efficiency of the operations as the equipment has to be shut down to remove the foulant, which results in production loss, cleaning expenditure, operation inconvenience, as well as related safety and environmental issues.

### SUMMARY

Described herein are compositions and methods for inhibiting or reducing fouling by salts of ammonium such as ammonium sulfate thereby improving energy efficiency of a system and preventing product quality issues.

In one aspect of the invention is a method of inhibiting deposition of a foulant comprising:

introducing into a process a composition comprising at least one sulfonated compound having the general structure:

R—(SO₃)ₙM

Wherein R is a hydrocarbon group chosen from a linear or branched alkyl, aromatic, cyclic, alkaryl, aralky, or alkenyl groups, and mixtures thereof;

M is H, alkali metals, alkaline earth metals, alkali metal cations, alkaline earth metal cations ammonium cations, alkyl ammonium cations, or mixtures thereof; and

n ranges from 1 to about 6.

In other aspects of the invention, is a composition comprising at least one sulfonated compound to inhibit deposition of a foulant in contact with process equipment, the at least one sulfonated compound comprising the general structure:

R—(SO₃)ₙM

wherein R is a hydrocarbon group chosen from a linear or branched alkyl, aromatic, cyclic, alkaryl, aralky, or alkenyl groups and mixtures thereof;

M is H, alkali metals, alkaline earth metals, alkali metal cations, alkaline earth metal cations, ammonium cations, alkyl ammonium cations, or mixtures thereof; and

n ranges from 1 to about 6.

In some aspects, the sulfonated compounds can include a sulfonated fatty acids, sulfated oils, sulfated fatty acids, naphthalene sulfonate formaldehyde condensate, naphthalene sulfonic acid copolymers, sulfonic acids, dodecylbenzene sulfonic acid, styrene sulfonate polymers, and lignosulfonate metal salts or combination thereof.

In some aspects the styrene polymer has the general structure:

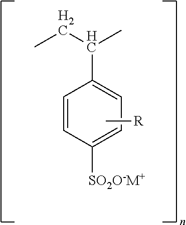

wherein M is hydrogen, alkali metals or ammonium or a mix of them, R is hydrogen, alkyl aryl, alkylaryl, arylalkyl, R may contain heteroatoms, n is an integer.

In still other aspects of the invention is a composition comprising:
a fluid; and
the at least one sulfonated compound.

The described sulfonated compounds can be used to inhibit fouling of ammonium salts, particularly in processes that concentrate the ammonium salts.

### BRIEF DESCRIPTION OF THE DRAWINGS

FIG. 1 is an illustration of an exemplary concentration process of ammonium sulfate.

FIG. 2 is a graphical representation showing the effects of embodiments of the invention on a foulant.

### DETAILED DESCRIPTION

Although the present disclosure provides references to various embodiments, persons skilled in the art will recognize that changes may be made in form and detail without departing from the spirit and scope of the invention. Various embodiments will be described in detail with reference to the figures. Reference to various embodiments does not limit the scope of the claims attached hereto. Additionally, any examples set forth in this specification are not intended to be limiting and merely set forth some of the many possible embodiments for the appended claims.

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present invention. All publications, patent applications, patents and other references mentioned herein are incorporated by reference in their entirety.

As used herein, the term “antifoulant” refers to a composition or compound that “inhibits” the formation or deposition of foulant on “process equipment.” The term will be understood to refer to the antifoulant itself or in a composition which may include other antifoulants or compounds or solvents, as determined by context.

The term “foulant” refers to a material that accumulates on process equipment during the operation of manufacturing or chemical processes that are unwanted and impair operation and efficiency of the processes. “Foulant” includes the formation of material such as polymers, prepolymers, oligomer and/or other materials such as ammonium salts such as ammonium sulfate, ammonium chloride, ammonium nitrate, which would become insoluble in and/or precipitate from a stream and deposit on process equipment under conditions of operating the process equipment.

As used herein, the term “inhibits,” “inhibiting,” or grammatical equivalents thereof refer to preventing, retarding, mitigating, reducing, minimizing, controlling and/or delaying the deposition of foulant.

As used herein, the term “process equipment” means equipment used to refine, store, transport, fractionate, or otherwise process a material including but not limited to heaters, heat exchangers, tubes, pipes, heat transfer vessels, process vessels, tanks, compressors, fans, impellers, pumps, valves, inter-coolers, sensors, strippers, quench towers or quench columns, evaporators, crystallizers, and the like, that are associated with a process and which may be subject to the deposition of foulant. This term also includes sets of components which are in communication such as, for example, a quench tower and evaporator in an ammonium sulfate process.

As used herein, the terms “comprise(s),” “include(s),” “having,” “has,” “can,” “contain(s),” and variants thereof are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of’ and “consisting essentially of,” the embodiments or elements presented herein, whether explicitly set forth or not.

As used herein, the term “optional” or “optionally” means that the subsequently described event or circumstance may but need not occur, and that the description includes instances where the event or circumstance occurs and instances in which it does not.

As used herein, the term “about” modifying, for example, the quantity of an ingredient in a composition, concentration, volume, process temperature, process time, yield, flow rate, pressure, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities. Further, where “about” is employed to describe a range of values, for example “about 1 to 5” the recitation means “1 to 5” and “about 1 to about 5” and “1 to about 5” and “about 1 to 5” unless specifically limited by context.

As used herein, the term “substantially” means “consisting essentially of” and includes “consisting of” “Consisting essentially of” and “consisting of” are construed as in U.S. patent law. For example, a solution that is “substantially free” of a specified compound or material may be free of that compound or material, or may have a minor amount of that compound or material present, such as through unintended contamination, side reactions, incomplete purification or test methods used. A “minor amount” may be a trace, an unmeasurable amount, an amount that does not interfere with a value or property, or some other amount as provided in context. A composition that has “substantially only” a provided list of components may consist of only those components, or have a trace amount of some other component present, or have one or more additional components that do not materially affect the properties of the composition. Additionally, “substantially” modifying, for example, the type or quantity of an ingredient in a composition, a property, a measurable quantity, a method, a value, or a range, employed in describing the embodiments of the disclosure, refers to a variation that does not affect the overall recited composition, property, quantity, method, value, or range thereof in a manner that negates an intended composition, property, quantity, method, value, or range. Where modified by the term “substantially” the claims appended hereto include equivalents according to this definition.

As used herein, any recited ranges of values contemplate all values within the range and are to be construed as support for claims reciting any sub-ranges having endpoints which are real number values within the recited range. By way of example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

Described herein are compositions and methods that use an antifoulant to inhibit or reduce the formation of ammonium salts such as ammonium sulfate as foulants. The antifoulant can include sulfonated compounds. In some embodiments, the sulfonated compounds are sulfonated oils, sulfonated fatty acids, sulfated oils, sulfated fatty acids, naphthalene sulfonate formaldehyde condensates, styrene sulfonate polymers and their associated salts thereof and mixtures and combinations thereof. In some embodiments, the sulfonated compounds disperse the foulant in ammonia-related processes. In some embodiments, the sulfonated compounds disperse the ammonium salts (e.g. ammonium sulfate) as foulant in ammonium processes.

In some embodiments, the sulfonated compounds suitable for use herein has the general structure:

R—(SO₃)ₙM

Wherein R is a hydrocarbon group chosen from a linear or branched alkyl, aromatic, cyclic, alkaryl, aralky, or alkenyl groups and mixtures thereof;

M is H, alkali metals, alkaline earth metal, alkali metal cations, alkaline earth metal cations, ammonium cations, alkyl ammonium cations, or mixtures thereof, and n ranges from 1 to about 6.

In some embodiments, the sulfonated compounds suitable for use herein has the general structure:

R—(SO₃M)ₙ

Wherein R is a hydrocarbon group chosen from a linear or branched alkyl, aromatic, cyclic, alkaryl, aralky, or alkenyl groups and mixtures thereof;

M is H, alkali metals, alkaline earth metal, alkali metal cations, alkaline earth metal cations, ammonium cations, alkyl ammonium cations, or mixtures thereof, and n ranges from 1 to about 6.

In yet other embodiments, the sulfonated compounds suitable for use herein has the general structure:

R—(SO3)ₙMₙ

Wherein R is a hydrocarbon group chosen from a linear or branched alkyl, aromatic, cyclic, alkaryl, aralky, or alkenyl groups and mixtures thereof;

M is H, alkali metals, alkaline earth metal, alkali metal cations, alkaline earth metal cations, ammonium cations, alkyl ammonium cations, or mixtures thereof, and n ranges from 1 to about 6.

In some embodiments R is a hydrocarbonaceous group having from 1-34 carbon atoms chosen from linear or branched alkyl groups, aromatic, cyclic, alkaryl, aralkyl, or alkenyl groups, alkyl diphenyl ether groups, dialkyl naphthalene groups, or mixtures thereof.

In some embodiments the sulfonated compounds are alkyl sulfonic acids, alkyl aromatic sulfonic acids or alkyl naphthenic sulfonic acids. In some embodiments the alkyl sulfonic acids are organic sulfonic acids such as toluene sulfonic acid, methane sulfonic acid, dodecyl sulfonsuccinic anhydride, dodecyl sulfosuccinic acid, and dioctyl sulfosuccinate.

In some embodiments the sulfonated compounds are naphthalene sulfonic acid-HCO copolymer and salts thereof or 2-naphtalenesulfonic acid-HCO copolymer and salts thereof.

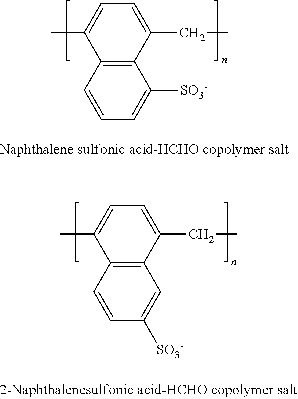

In some embodiments the sulfonated compounds are dodecylbenzene sulfonic acid, methyl sulfonic acid, toluene sulfonic acid, alkyldiphenyl ether disulfonic acid, dialkyl naphthalene sulfonic acid, dioctyl sulfosuccinic acid, and mixtures thereof.

In some embodiments the sulfonated compounds are neutralized, polymeric condensation products of naphthalene sulfonic acid and formaldehyde. These naphthalene sulfonate formaldehyde condensates may extend from a molecular weight of about X to about Y. The naphthalene portion may be sulfonated in either the 1 or 2 position. Methylene linkages typically connect the sulfonated naphthalene rings at positions 5 or 8. The polymers may be neutralized with a variety of bases or mixture of bases including sodium, potassium, calcium, and ammonium hydroxide. The general structure of the naphthalene sulfonate formaldehyde condensate is —CH₂[C₁₀H₅(SO₃M)]ₙ-, where M may be Na⁺, K⁺, Ca⁺², NH₄⁺, or the like. In some embodiments, the naphthalene sulfonate formaldehyde condensate is of a molecular weight from about 1000 to about 1 million Daltons and is salts of sodium, potassium, calcium, ammonium hydroxide, and/or mixtures thereof. In other embodiments, the naphthalene sulfonate formaldehyde condensate is of a molecular weight from about 2500 to about 500,000 Daltons, or from about 3000 to about 10,000 Daltons.

Any method known to one of skill in the art may be used to prepare the sulfonated compounds. For example, sulfonated oils, sulfonated fatty acids, sulfated oils, sulfated fatty acids, naphthalene sulfonate formaldehyde, sulfonic acids, dodecylbenzene sulfonic acid, and lignosulfonate metal salts, and sulfonate polymers have been described in U.S. Pat. Nos. 5,650,072, 5,746,924, 3,691,226, and 8,067,629, and each is incorporated herein by reference in their entireties.

In some embodiments the sulfonated compound is a styrene sulfonate polymer. In some embodiments, the polymeric material has the following repeating units:

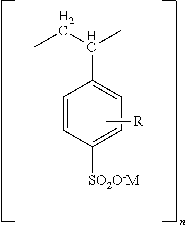

wherein M is hydrogen, alkali metals or ammonium or a mix of them, R is hydrogen, alkyl aryl, alkylaryl, arylalkyl, R may contain heteroatoms, n is an integer.

In some embodiments the styrene sulfonate polymer has a molecular weight of 50,000 to 2,000,000 or at least 100,000 to 1,000,000 Dalton.

Various solvents may be used to prepare the sulfonated compound such as alcohols, ethers, esters, ketones, nitriles or mixtures thereof. In some embodiments, organic polar solvents (protic and aprotic) are used such as butyl cellosolve or any of the ethylene oxide based cellosolve capped ether solvents, and may also include such organic polar solvents as the diethyl ether of tetraethylene glycol, polyethylene and polypropylene oxide alkyl ethers, and generally may also include other ether solvents, such as diethyl ether. In addition, other polar solvents that also function include certain organic acids, such as acetic acid, or such other polar solvents such as diacetone alcohol, linear alkyl and branched alkyl alcohols, such as methanol, ethanol, propanol, isopropanol, t-butyl alcohol, and the like. Admixtures of these polar solvents may also be used.

Other solvents that may be used include esters, such as ethyl acetate, ketones, such as acetone, nitrites, such as acetonitrile and acrylonitrile, water (when blended with some of the above solvents), and admixtures of the above solvents. Also included are aliphatic and aromatic hydrocarbon solvents, dimethylacetmide (DMAC), dimethylformamide (DMF), dimethyl sulfoxide (DMSO), and heavy aromatic naphtha.

For example, the sulfonated compounds can be prepared in water. Co-solvents may also be used with water to enhance the solubility and improve product stability and handling. In some embodiments, styrene sulfonate polymer as the sulfonated compound is prepared in water.

In some embodiments the sulfonated compounds are prepared as a stock composition dissolved in a solvent to a concentration of about at least about 0.01% (wt), at least about 50% (wt), or in an amount in the range from about 0.01% (wt) to about 100% (wt).

An amount of the stock composition including the sulfonated compounds can be added to a composition or process stream or a composition or process stream capable of forming the foulant, to provide the antifoulant at a concentration effective to inhibit or reduce the deposition of the foulant (e.g. ammonium salts). In some embodiments the process stream can include HCN, acetonitrile and heavy nitriles, corrosion products, polymers, catalyst fines, and ammonium salts.

While an effective amount of the sulfonated compound used depends on a number of factors such as the local operating conditions, the temperature and other characteristics of the process, the stream containing the foulant (e.g. ammonia salts) to be processed, in some embodiments, the sulfonated compound or in compositions are used in an amount from about 0.1 ppm to 10,000 ppm; from 0.1 ppm to 3,000 ppm; from about 100 ppm to 1000 ppm; from about 500 ppm to 3,000 ppm; from about 750 ppm to 3,000 ppm; from about 2,000 ppm to 5,000 ppm; from about 3,000 ppm to 5000 ppm; from about 100 ppm to 3,000 ppm; from 50 ppm to 2000 ppm; from about 1 ppm to 1000 ppm; from about 1 ppm to 3,000 ppm; from about 10 ppm to 50 ppm; from about 50 ppm to 100 ppm, from 100 pp to 800 ppm, from 150 ppm to 550 ppm; from about 1 ppm to 250 ppm; from about 1 ppm to 50 ppm; from about 1 ppm to 25 ppm; from about 1 ppm to 5 ppm; from about 3 ppm to 25 ppm; from 0.1 ppm to 5 ppm; or from about 0.1 ppm to 1 ppm by weight or volume of the sulfonated compound in a fluid source.

In some embodiments, the compositions comprise, consist essentially of, or consist of at least one of the described sulfonated compounds. The sulfonated compounds can be formulated as an antifoulant composition useful to inhibit deposition of foulant (e.g. ammonium salts) on metal surfaces of process equipment in contact with an ammonia (in either liquid or gaseous form) which surfaces or liquid reach temperatures from 10° C. to 110° C.

In some embodiments the sulfonated compound is part of a composition including other antifoulants or dispersants, polymerization inhibitors, corrosion inhibitors, emulsifiers, water clarifiers, emulsion breakers, or any combination thereof.

In some embodiments the sulfonated compound is used in a method for inhibiting or reducing the formation of foulant such as ammonium salts and other organic matters. In some embodiments the ammonium salts are ammonium sulfate (NH₄)₂SO₄, ammonium chloride (NH₄Cl), ammonium nitrate (NH₄NO₃), ammonium dihydrogen phosphate (NH₄H₂PO₄), diammonium phosphate (NH₄)₂HPO₄, ammonium phosphate (NH₄)₂HPO₄) or mixtures thereof. In some embodiments the sulfonated compounds are used as an antifoulant to disperse the ammonium salts generated in an ammonium recovery or concentration system. In some embodiments the antifoulant is a naphthalene sulfonic acid polymer or condensates or a styrene sulfonate polymer or combinations thereof.

In some embodiments the foulant includes HCN, acetonitrile and heavy nitriles, corrosion products, polymers, catalyst fines, and ammonium salts. In some embodiments the foulant can be present in process water from about 75 wt % water, about 15 wt % ammonium salts, about 8.2 wt % polymers, 0.9 wt % acrylonitrile and 0.3 wt % catalyst fine dust. In some embodiments the antifoulant is a naphthalene sulfonic acid polymer or condensates or a styrene sulfonate polymer or combinations thereof used for a sample containing the foulant, which includes HCN, acetonitrile and heavy nitriles, corrosion products, polymers, catalyst fines, and ammonium salts.

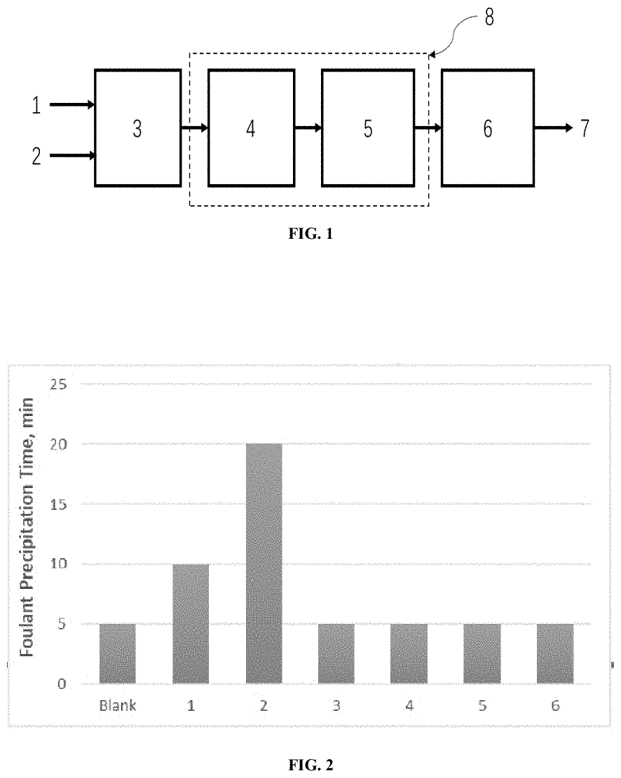

In some embodiments, a general commercial process for concentrating ammonium salts (e.g. ammonium sulfate) as a by-product or to reduce the volume of waste is shown in FIG. 1. Referring to FIG. 1, a process stream containing residual ammonia 2, originating from an industrial process, is quenched in an absorber 3 with stream 1 containing sulfate acid to produce aqueous ammonium sulfate. The aqueous ammonium sulfate is concentrated by passing it to evaporator 4. The concentrated ammonium sulfate is then passed into crystallizer 5 to form ammonium sulfate crystals. The ammonium sulfate crystal is separated from the mother liquor in the separator 6 to form ammonium sulfate product 7.

In some embodiments, the antifoulant (e.g. sulfonated compounds) is applied in the evaporation/concentration system 8 including evaporator 4, crystallizer 5 units and their auxiliaries like pipeline and pumps.

In some embodiments effluent containing ammonium salts is concentrated AND separated in several stages, which includes precipitation crystallization and the precipitated salt (e.g. ammonium sulfate) at the same time returned to the stage of concentration by evaporation. The residual mother liquor discharged from the final stage of evaporation is optionally subjected to a vacuum cooling crystallization at 10° to 80° C., from about 35° to 60° C., and the ammonium sulfate formed is separated off and can be returned to the evaporation crystallization. Dilution with water is carried out before the precipitation crystallization, optionally after a vacuum cooling crystallization. Ammonia is added to the resulting solution at a pressure of up to about 5 kg/cm², from about 1 to 2 kg/cm², the ammonium sulfate which has crystallized out is separated from the ammoniacal mother liquor, optionally washed free from ammonia and can be returned to the evaporation crystallization stage and the ammonia is then recovered from the ammoniacal mother liquor by distillation. An example of a process for treating ammonium sulfate is described in U.S. Pat. No. 4,292,043, which patent is incorporated by reference herein in its entirety.

In some embodiments concentration systems for ammonium salts (e.g. ammonium sulfate) include more than one evaporator. In some embodiments the multiple stage evaporation process can include at least two evaporators a circulating pump, a steam heater, a condenser and an ammonium sulfate solution tank or stream; the evaporator is for concentrating the ammonium sulfate from various sources such as an acrylonitrile unit and includes a first evaporator and a second evaporator, wherein the first evaporator is used for directly processing the ammonium sulfate from an acrylonitrile unit, the second evaporator is used for processing the processed ammonium sulfate from the first evaporator, and the first evaporator is communicated with the second evaporator simultaneously through a steam pipeline and an ammonium sulfate solution conveying pipeline; the circulating pump is used for circulating the ammonium sulfate solution; the steam heater is used for heating the medium in the evaporator; the condenser is used for condensing the steam discharged by the evaporator; the ammonium sulfate solution tank is used for collecting a concentrated solution; the steam heater is connected with the first-effect evaporator; and the condenser and the ammonium sulfate solution tank are respectively communicated with the second-effect evaporator. The double-effect evaporation system can improve the energy utilization efficiency, reduce steam consumption and operating cost, and improve environmental and economic benefits.

In some embodiments, the concentration system for ammonium-sulfate is part of an acrylonitrile system. In some embodiments, the ammonium-sulfate concentration system is part of a propylene-ammoxidation-reaction type acrylonitrile system. When acrylonitrile is made by the catalytic ammoxidation of propylene, propane, isobutene or isobutylene in the production of acrylonitrile or methacrylonitrile, ammonia is used. In other embodiments, the ammonium-sulfate concentration system is part of a coke oven gas quenching system in a coking plant. The unreacted or residual ammonia can be recycled or concentrated in processes as described above. An example of a process for treating ammonium sulfate is described in Chinese patent application no. CN203108242U, which is incorporated by reference herein in its entirety. In other embodiments, other variations of ammonium salt concentration systems are described in Chinese patent application no. CN106075943AU. In some embodiments, the sulfonated compound is applied in the pipelines that connect multiple evaporators or other parts of the system.

In some embodiments the sulfonated compound is introduced into one or more fluid streams of the quench column and the recovery stage as well as the wastewater process where the sulfonated compound acts as a dispersant that prevents foulant deposition and even facilitates the removal of previously deposited foulant. In some embodiments the sulfonated compound is introduced

In some embodiments the antifoulant is a sulfonated compound used to inhibit or reduce the formation of ammonium salts such as ammonium sulfate as the foulant in an ammonium sulfate concentration system, which concentration system is part of an acrylonitrile system. Examples of acrylonitrile plants are described in U.S. Pat. Nos. 5,650,072, 5,746,924, 3,691,226, and 8,067,629, each incorporated by reference in their entireties.

The sulfonated compound may be added by any suitable method. For example the sulfonated compound may be added neat or as a dilute solution. In some embodiments, the sulfonated compound may be introduced as a solution, emulsion, or dispersion that is sprayed, dripped, poured or injected into a desired opening within a system or onto the process equipment.

The sulfonated compound can be added continuously or intermittently to the process equipment as required to inhibit fouling. In some embodiments, the sulfonated compound can be pumped or injected into a system in a continuous fashion or as an intermittent fashion to mitigate the fouling in the process unit. The injection point can be at any or all stages of the process unit.

The sulfonated compound is used on any suitable process equipment. In some embodiments, the process equipment includes thermal conversion units, heat exchanger, visbreakers, cokers, fired heaters, furnaces, fractionators, or other heat transfer equipment. In some embodiments the process equipment is gas compressors. In some embodiments, the process equipment is coils, heat exchangers, transfer line exchangers quench coolers or quench towers or columns, furnaces, separation columns or fractionators, evaporators and crystallizers. The sulfonated compound can also be useful in other similar applications and with other equipment. For example, the sulfonated compound may be used with any process where process equipment will come into contact with ammonium salts such as ammonium sulfate or ammonium chloride. In some embodiments the process is ethylene and acrylonitrile quench water systems. The sulfonated compound may be used with ethylene dilution steam generators and acrylonitrile purification systems. Many polymer processes have monomer recovery systems which are subject to fouling and are good target applications for the sulfonated compounds. The sulfonated compound can be used in any process which has process equipment subject to foulant (e.g. ammonium salts) forming and depositing on process equipment.

In some embodiments the sulfonated compound is introduced into a fluid by any means suitable for ensuring dispersal of the sulfonated compound through the fluid source being treated. The composition comprising the sulfonated compound can be injected as prepared or formulated in one or more additional solvents, depending upon the application and requirements. One of skill in the art will understand that the methods disclosed herein are not limited in any way by the introduction method, the timing or the location of the introduction.

In some embodiments the sulfonated compound is introduced into process equipment or fluid in contact with the process equipment. In some embodiments, the process equipment is used to refine, store, transport, fractionate, or otherwise process an ammonium stream with salts such as sulfate or chloride.

The sulfonated compound or in compositions is introduced into process equipment to form treated process equipment. In some embodiments, treated process equipment can be observed to undergo less foulant deposition than on process equipment without addition of the sulfonated compound or in compositions.

Inhibition in the foulant formation or foulant deposition can be evaluated by any known method or test. In some embodiments, the inhibition of foulant formation and foulant deposition on process equipment can be assessed by measuring the time of it takes to disperse the foulant as described in Example 1.

The sulfonated compound or in compositions can be used for any process equipment having a metal surface. In some embodiments, the metal surface of the process equipment is metal or metal alloys. For example, the metal surface can include steel (including carbon steel, stainless steel, galvanized steel, hot dipped galvanized steel, electrogalvanized steel, annealed hot dipped galvanized steel, or mild steel), nickel, titanium, tantalum, aluminum, copper, gold, silver, platinum, zinc, nickel titanium alloy (nitinol), an alloy of nickel, chromium, iron, iridium, tungsten, silicon, magnesium, tin, alloys of any of the foregoing metals, coatings containing any of the foregoing metals, and combinations thereof. In some embodiments, the metal surface of the process equipment is iron alloys, carbon steel, stainless steel, nickel-chromium-iron alloys, or other alloys.

In some embodiments, the deposition of foulant process equipment treated with the sulfonated compound is reduced by at least 50 wt % compared to process equipment not treated with the sulfonated compound. In some embodiments, about 50 wt % to 100 wt % (where 100 wt % reduction in foulant formation is elimination of deposition), or about 50 wt % to 95 wt %, or about 50 wt % to 90 wt %, or about 50 wt % to 85 wt %, or about 50 wt % to 80 wt %, or about 50 wt % to 75 wt %, or about 50 wt % to 70 wt %, or about 55 wt % to 100 wt %, or about 60 wt % to 100 wt %, or about 65 wt % to 100 wt %, or about 70 wt % to 100 wt %, or about 60 wt % to 95 wt %, or about 70 wt % to 95 wt %, or about 60 wt % to 90 wt %, or about 70 wt % to 90 wt %.

Some additional non-limiting embodiments are provided below to further exemplify the present disclosure:

Embodiment 1: A method of inhibiting deposition of a foulant comprising:

introducing into a process a composition comprising at least one sulfonated compound having the general structure:

R—(SO₃M)ₙ

Wherein R is a hydrocarbon group chosen from a linear or branched alkyl, aromatic, cyclic, alkaryl, aralky, or alkenyl groups, and mixtures thereof;

M is H, alkali metals, alkaline earth metals, alkali metal cations, alkaline earth metal cations, ammonium cations, alkyl ammonium cations, or mixtures thereof; and

n ranges from 1 to about 6.

Embodiment 2: A method of inhibiting deposition of a foulant comprising:

introducing into a process a composition comprising at least one sulfonated compound having the general structure:

R—(SO₃)ₙMₙ

Wherein R is a hydrocarbon group chosen from a linear or branched alkyl, aromatic, cyclic, alkaryl, aralky, or alkenyl groups, and mixtures thereof;

M is H, alkali metals, alkaline earth metals, alkali metal cations, alkaline earth metal cations, ammonium cations, alkyl ammonium cations, or mixtures thereof; and

n ranges from 1 to about 6.

Embodiment 3 The method as in any one of embodiments 1-2, wherein the introducing is by injecting, spraying, or dripping the sulfonated compound.

Embodiment 4: The method as in any one of embodiments 1-3, wherein the introducing is carried out during or after cleaning or during the process.

Embodiment 5: The method as in any one of embodiments 1-4, wherein the process is an ammonium concentration process.

Embodiment 6: The method as in any one of embodiments 1-5, wherein the process is an ammoxidation reaction of propylene, propane, isobutene, or isobutylene.

Embodiment 7: The method as in any one of embodiments 1-6, wherein the process is an acrylonitrile process.

Embodiment 8: The method as in any one of embodiments 1-7, wherein the introducing is carried out intermittently.

Embodiment 9: The method as in any one of embodiments 1-8, wherein the introducing is carried out continuously.

Embodiment 10: The method as in any one of embodiments 1-9, wherein the process equipment comprises coils, heat exchangers, transfer line exchangers quench coolers or towers, furnaces, separation columns fractionators, evaporators, crystallizers, or combinations thereof.

Embodiment 11: The method as in any one of embodiments 1-10, wherein the process equipment comprises evaporators, crystallizers of an ammonium concentration system

Embodiment 12: The method as in any one of embodiments 1-11, wherein the foulant comprises HCN, acetonitrile and heavy nitriles, corrosion products, polymers, catalyst fines, and ammonium salts.

Embodiment 13: The method as in any one of embodiments 1-12, wherein the foulant comprises ammonium sulfate, ammonium chloride, or mixtures thereof.

Embodiment 14: The method as in one of embodiments 1-13, wherein the at least one sulfonated compound is added to a fluid in the process from 1 ppm to 3000 ppm of the fluid volume.

Embodiment 15: The method as in one of embodiments 1-14, wherein the composition further comprises one or more other antifoulants or dispersants, polymerization inhibitors, corrosion inhibitors, emulsifiers, or any combination thereof.

Embodiment 16: The method as in one of embodiments 1-15, wherein the at least one sulfonated compound comprises sulfonated fatty acids, sulfated oils, sulfated fatty acids, naphthalene sulfonate formaldehyde, naphthalene sulfonic acid copolymers, sulfonic acids, dodecylbenzene sulfonic acid, styrene sulfonate polymers, and lignosulfonate metal salts or combination thereof.

Embodiment 17: The method as in one of embodiments 1-16, wherein the at least one sulfonated compound comprises a styrene sulfonate polymer having the general structure:

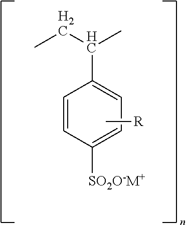

wherein M is hydrogen, alkali metals or ammonium or a mix of them, R is hydrogen, alkyl aryl, alkylaryl, arylalkyl, R may contain heteroatoms, n is an integer.

Embodiment 18: The method as in one of embodiments 1-17, wherein the at least one sulfonated compound is a styrene sulfonate polymer having a molecular weight of 50,000 to 2,000,000 Daltons, a naphthalene sulfonate formaldehyde condensate having a molecular weight of 1000 to about 1 million Daltons or combination thereof.

Embodiment 19: The method as in one of embodiments 1-18 wherein the at least one sulfonated compound provides 50%-95% inhibition of foulant deposition.

Embodiment 20: The method as in one of embodiments 1-19, wherein the at least one sulfonated compound provides 50%-95% inhibition of foulant deposition in a dispersancy test.

Embodiment 21: The method as in one of embodiments 1-20, wherein the introducing the at least one sulfonated compound inhibits foulant deposition of the process equipment compared to a process equipment under the same conditions without the introduction of the sulfonated compound.

Embodiment 22: A composition comprising at least one sulfonated compound to inhibit deposition of a foulant in contact with process equipment, the at least one sulfonated compound comprising the general structure:

R—(SO₃M)ₙ

wherein R is a hydrocarbon group chosen from a linear or branched alkyl, aromatic, cyclic, alkaryl, aralky, or alkenyl groups and mixtures thereof;

M is H, alkali metals, alkaline earth metals, ammonium cations, alkyl ammonium cations, or mixtures thereof; and
n ranges from 1 to about 6.

Embodiment 23: A composition comprising at least one sulfonated compound to inhibit deposition of a foulant in contact with process equipment, the at least one sulfonated compound comprising the general structure:

R—(SO3)ₙMₙ
wherein R is a hydrocarbon group chosen from a linear or branched alkyl, aromatic, cyclic, alkaryl, aralky, or alkenyl groups and mixtures thereof,
M is H, alkali metals, alkaline earth metals, ammonium cations, alkyl ammonium cations, or mixtures thereof, and
n ranges from 1 to about 6.

Embodiment 24: The composition as in one of embodiments 22-23, wherein the foulant comprises at least ammonium salts.

Embodiment 25: The composition as in one of embodiments 22-24, wherein the foulant comprises at least ammonium sulfate (NH4)2SO4, ammonium chloride (NH4Cl), ammonium nitrate (NH4NO3), ammonium dihydrogen phosphate (NH4H2PO4), diammonium phosphate (NH4)2HPO4, ammonium phosphate (NH4)2HPO4) or mixtures thereof.

Embodiment 26: The composition as in one of embodiments 22-25, wherein the at least one sulfonated compound comprises a sulfonated fatty acids, sulfated oils, sulfated fatty acids, naphthalene sulfonate formaldehyde, naphthalene sulfonic acid copolymers, sulfonic acids, dodecylbenzene sulfonic acid, styrene sulfonate polymers, and lignosulfonate metal salts or combination thereof.

Embodiment 27: The composition as in one of embodiments 22-26, wherein the at least one sulfonated compound comprises a styrene sulfonate polymer having the general structure:

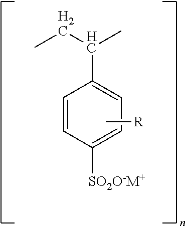

wherein M is hydrogen, alkali metals or ammonium or a mix of them, R is hydrogen, alkyl aryl, alkylaryl, arylalkyl, R may contain heteroatoms, n is an integer.

Embodiment 28: The composition as in one of embodiments 22-27, wherein the sulfonated compound is about 1 ppm to 3000 ppm of the composition.

Embodiment 29: The composition of as in one of embodiments 22-28, wherein the composition further comprises one or more other antifoulants or dispersants, polymerization inhibitors, corrosion inhibitors, emulsifiers, or any combination thereof.

Embodiment 30: A composition comprising:
a fluid; and
the at least one sulfonated compound as in one of embodiments 22-29.

Embodiment 31: The composition of embodiment 30, wherein the fluid is in contact with coils, heat exchangers, transfer line exchangers quench coolers or towers, furnaces, separation columns fractionators, evaporators, crystallizers, or combinations thereof.

Embodiment 32: The composition as in one of embodiments 30-31, wherein the fluid comprises at least ammonium salts.

Embodiment 33: The composition as in one of embodiments 30-31, wherein the fluid comprises at least ammonium sulfate, ammonium chloride or mixtures therefor.

Embodiment 34: The composition as in one of embodiments 30-33, wherein the fluid temperature is about 10° C. to 101° C.

Embodiment 35: A treated process equipment comprising:

a process equipment comprising a metal surface; and

the fluid source comprising the sulfonated compound as in one of embodiments 21-31, wherein at least a portion of the metal surface is contacted by the fluid source.

Embodiment 36: The treated process equipment of embodiment 35, wherein the process equipment comprises iron or iron alloys.

Embodiment 37: The treated process equipment of embodiment 36, wherein the iron alloys comprise carbon steel, stainless steel, nickel-chromium-iron alloys, or other alloys.

Embodiment 38: The treated process equipment as in one of embodiments 35-37, wherein the metal containment comprises coils, heat exchangers, transfer line exchangers quench coolers or towers, furnaces, separation columns fractionators, evaporators, crystallizers, or combinations thereof.

Embodiment 39: The treated process equipment as in one of embodiments 35-38, wherein the fluid comprises, foulant comprising at least ammonium salts.

Embodiment 40: The treated process equipment as in any one of embodiments 35-39, wherein the foulant comprises ammonium sulfate, ammonium chloride, or mixtures thereof.

Embodiment 41: Use of the sulfonated compound as in one of embodiments 1-40 to inhibit fouling of ammonium salts.

### EXAMPLES

The following examples are intended to illustrate different aspects and embodiments of the invention and are not to be considered limiting the scope of the invention. It will be recognized that various modifications and changes may be made without following the experimental embodiments described herein, and without departing from the scope of the claims.

### Example 1

To determine the effectiveness of various dispersants, a sample of an ammonium sulfate foulant, which includes catalyst fine dust, and polymers was taken from an ammonium sulfate evaporation/concentration system in an acrylonitrile plant. The foulant sample was dried and ground to powder. The various dispersants were dissolved in either water or a polar solvent.

Various dispersants as shown in Table 1 were each added into separate test tubes. Each test tube contained 10 ml process water (75.5 wt % water, 15.4 wt % ammonium sulfate, 8.2 wt % polymers, 0.9 wt % acrylonitrile and 0.3 wt % other materials including catalyst fines), 0.05 grams from ammonium sulfate foulant and various doses of the dispersants. The contents of each tube was mixed and allowed to stand at ambient temperature (e.g. to 25-30° C.) The data is reported as the time it took for the contents in the tube to precipitate. A test tube with all the above-described contents but without a dispersant served as a blank. The dosage for the various dispersants tested was 200 ppm, 400 ppm, 800 ppm and 1600 ppm.

The various dispersants tested are shown in Table 1. The results are the time it takes for the contents in each tube to precipitate for each dispersant tested and is shown in FIG. 2. The longer the time to precipitate, the better dispersing performance of the dispersant.

TABLE 1

| No. | Dispersant | Dosage |
|---|---|---|
| 1 | napthalenesulfonic | 50 ppm~1600 ppm |
| | acid copolymer salt) | |
| 2 | styrene sulfonate | 50 ppm~1600 ppm |
| | polymer | |
| 3 | polyacrylate | 50 ppm~1600 ppm |
| | copolymer | |
| 4 | sodium salt of | 50 ppm~1600 ppm |
| | phosphonomethylated | |
| | diamine and acrylate | |
| | polymer | |
| 5 | carboxylate) | 50 ppm~1600 ppm |
| 6 | anionic acrylic | 50 ppm~1600 ppm |
| | polymer | |

As seen in FIG. 2 above, the styrene sulfonate polymer (2) and napthalenesulfonic acid copolymer salts (1) showed the best ability to disperse the foulant compared to the blank and the other tested dispersants.

1. A method of inhibiting deposition of a foulant comprising:
introducing into a process a composition comprising at least one sulfonated compound having the general structure:
R—(SO₃)ₙM
wherein R is a hydrocarbon group chosen from a linear or branched alkyl, aromatic, cyclic, alkaryl, aralky, or alkenyl groups, and mixtures thereof;
M is H, alkali metals, alkaline earth metals, alkali metal cations, alkaline earth metal cations ammonium cations, alkyl ammonium cations, or mixtures thereof; and
n ranges from 1 to about 6.

2. The method of claim 1, wherein the introducing is by injecting, spraying, or dripping the sulfonated compound.

3. The method of claim 1, wherein the introducing is carried out during or after cleaning or during the process.

4. The method of claim 1, wherein the process is an ammonium concentration process.

5. The method of claim 1, wherein the process is an ammoxidation reaction of propylene, propane, isobutene, or isobutylene.

6. The method of claim 1, wherein the process is an acrylonitrile process.

7. The method of claim 1, wherein the introducing is carried out intermittently or continuously.

8. (canceled)

9. The method of claim 1, wherein the process equipment comprises coils, heat exchangers, transfer line exchangers, quench coolers, quench towers, furnaces, separation columns fractionators, evaporators, crystallizers, or combinations thereof.

10. (canceled)

11. The method of claim 1, wherein the foulant comprises HCN, acetonitrile and heavy nitriles, corrosion products, polymers, catalyst fines, and ammonium salts.

12. (canceled)

13. The method of claim 1, wherein the at least one sulfonated compound is added to a fluid in the process from 1 ppm to 3000 ppm of the fluid volume.

14. The method of claim 1, wherein the composition further comprises one or more other antifoulants, one or more dispersants, polymerization inhibitors, corrosion inhibitors, emulsifiers, or any combination thereof.

15. The method of claim 1, wherein the at least one sulfonated compound comprises sulfonated fatty acids, sulfated oils, sulfated fatty acids, naphthalene sulfonate formaldehyde, naphthalene sulfonic acid copolymers, sulfonic acids, dodecylbenzene sulfonic acid, styrene sulfonate polymers, lignosulfonate metal salts, or combination thereof.

16. The method of claim 1, wherein the at least one sulfonated compound comprises a styrene sulfonate polymer having the general structure: 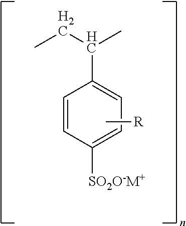
wherein M is hydrogen, alkali metals or ammonium or a mix of them, R is hydrogen, alkyl aryl, alkylaryl, arylalkyl, R may contain heteroatoms, n is an integer.

17. (canceled)

18. (canceled)

19. (canceled)

20. (canceled)

21. A composition comprising at least one sulfonated compound to inhibit deposition of a foulant in contact with process equipment, the at least one sulfonated compound comprising the general structure:
R—(SO₃)ₙM
wherein R is a hydrocarbon group chosen from a linear or branched alkyl, aromatic, cyclic, alkaryl, aralky, or alkenyl groups and mixtures thereof;
M is H, alkali metals, alkaline earth metals, alkali metal cations, alkaline earth metal cations ammonium cations, alkyl ammonium cations, or mixtures thereof; and
n ranges from 1 to about 6.

22. The composition as in claim 21, wherein the foulant comprises at least ammonium salts.

23. (canceled)

24. The composition of claim 21, wherein the at least one sulfonated compound comprises a sulfonated fatty acid, sulfated oils, sulfated fatty acids, naphthalene sulfonate formaldehyde, naphthalene sulfonic acid copolymers, sulfonic acids, dodecylbenzene sulfonic acid, styrene sulfonate polymers, lignosulfonate metal salts, or any combination thereof.

25. (canceled)

26. The composition of claim 21, wherein the sulfonated compound is about 1 ppm to 3000 ppm of the composition.

27. (canceled)

28. (canceled)

29. (canceled)

30. (canceled)

31. (canceled)

32. (canceled)

33. A treated process equipment comprising:
a process equipment comprising a metal surface; and
the fluid source comprising the sulfonated compound of claim 21, wherein at least a portion of the metal surface is contacted by the fluid source.

34. The treated process equipment of claim 33, wherein the process equipment comprises iron or iron alloys.

35. (canceled)

36. (canceled)

37. (canceled)

38. The treated process equipment of claim 33, wherein the foulant comprises ammonium sulfate, ammonium chloride, or mixtures thereof.

39. (canceled)
